(19) United States
(12) Patent Application Publication
(10) Pub. No.: US 2008/0293110 A1
(43) Pub. Date: Nov. 27, 2008

(54) Process for Isolation of Mycophenolic Acid

(75) Inventors: Ladislav Cvak, Opava (CZ); Jiri Faustmann, Opava (CZ); Josef Satke, Opava (CZ)

Correspondence Address: LERNER, DAVID, LITTENBERG,;KRUMHOLZ & MENTLIK, 600 SOUTH AVENUE WEST, WESTFIELD, NJ, 07090, US

(73) Assignee: IVAX Pharmaceuticals s.r.o., Opava 9 (CZ)

(21) Appl. No.: 11/662,231

(22) PCT Filed: Sep. 9, 2005
(86) PCT No.: PCT/US05/32259

§ 371 Date: Apr. 1, 2008

Related U.S. Application Data
(60) Provisional application No. 60/608,753, filed on Sep. 10, 2004.

(51) Int. Cl. C12P 17/04

(52) U.S. Cl. 435/126

(57) ABSTRACT

Mycophenolic acid can be isolated from fermentation broth easily with low consumption or organic solvents to produce mycophenolic acid that is surprisingly high in purity. The process can be accomplished by addition of a suitable base to the whole fermentation broth. i.e., a suspension obtained by submerged cultivation of a microorganism producing mycophenolic acid, to increase pH of the liquid phase to the value from about (9) to about (13). Mycophenolic acid is thus extracted from the mycelium to the liquid phase and the exhausted mycelium can be separated easily by filtration.

### FIELD OF INVENTION

The invention relates to a process for isolation of the immunosuppressant agent, mycophenolic acid of the Formula I, from the fermentation broth obtained by submerged cultivation of a strain producing mycophenolic acid, e.g. microorganisms of genus Penicillium or Eupenicillium.

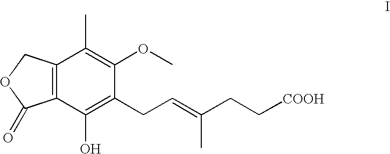

### BACKGROUND OF THE INVENTION

Mycophenolic acid was first described as a secondary metabolite of Penicillium glaucum (B. Gosio, Riv. Igiene Sanita Pub. Ann., 7, 825, (1896)). Its biological activities were discovered much later: antibacterial (E. P. Abraham et al., Biochem. J. 39, 398 (1945) and K. Gilliver, Ann. Bot. (London), 10, 271 (1946)), antiviral (R. H. Williams et al., J. Antibiot., 21, 463, (1968) and K. Ando et al., J. Antibiot., 21, 649 (1968)) and anticancer (Y. Sidi et al., Br. J. Cancer, 58, 61 (1988)). The main activity of mycophenolic acid, the immunosuppressant action, is associated with its interaction with purine metabolism: mycophenolic acid is a competitive reversible inhibitor of inosine monophosphate dehydrogenase as reviewed e.g. in Drugs Fut., 20, 356 (1995).

Mycophenolic acid is produced by several species of Penicillium and Eupenicillium, including P. brevicompactum, P. stoloniferum, P. scarbum, P. griseobrunneum, P. viridicatum. Numerous fermentation processes and producing strains are described in the patent literature e.g. GB 1,157,099, GB 1,593,208, U.S. Pat. No. 4,452,891, WO 03/106690, WO 01/21607. On the other hand, the isolation of the mycophenolic acid from the fermentation broth has been described only in one patent; WO 01/64931. The process further includes purification of the solution of mycophenolic acid on alumina and double crystallization from organic solvents.

### DETAILED DESCRIPTION OF THE INVENTION

The separated liquid phase is then acidified by addition of a suitable acid to pH from about 4.5 to about 1.5 to precipitate the mycophenolic acid from the solution. Mycophenolic acid is then separated dried and recrystallized from toluene to obtain product with purity higher than 99%.

### EXAMPLES

The following example is intended to further illustrate certain preferred embodiment of the invention and is not limiting in nature. Those skilled in the art will recognize, using no more than routine experimentation, numerous equivalents to the specific procedures described herein.

### Example 1

8000 liters of the whole fermentation broth obtained by submerged cultivation of the strain IJ69 of Penicillium brevicompactum, containing according to the HPLC analysis 5.254 g/l of mycophenolic acid, was alkalized by addition of 10% aqueous solution to pH 10.5 and the suspension was stirred for 3 hours. Solid particles were then filtered off on a rotary vacuum filter, using diatomaceous earth as a filtration aid. The filter cake was washed with 0.2% aqueous solution of sodium hydroxide. The clear filtrate was acidified by addition of 38% sulphuric acid to pH 3.0. Acidification causes mycophenolic acid to precipate and the precipitated mycophenolic acid was filtered off on a plate filter, washed with water adjusted to pH about 3 and dried in a vacuum dryer at 60° C. Crude mycophenolic acid was then dissolved in 600 liters of hot toluene, the insoluble part was filtered of and the clear solution was crystallized by cooling to about −5° C. The crystalline product was filtered on a nutsch filter, washed with toluene and dried in vacuum dryer at 60° C. 36.9 kg of product, containing according to HPLC analysis 99.2% of mycophenolic acid was obtained.

What we claimed is:

1. A process for isolation of mycophenolic acid from fermentation broth comprising
a) addition of a suitable base to whole fermentation broth to obtain a suspension with pH from 9 to 13,
b) separation of mycelium from the liquid phase,
c) addition of a suitable acid to the separated liquid phase to obtain an aqueous suspension of mycophenolic acid,
d) separation of the mycophenolic acid from the liquid phase,
e) dissolution of the separated mycophenolic acid in toluene,
f) crystallization of mycophenolic acid from the toluene solution.

2. The process of claim 1, wherein the whole fermentation broth is a suspension obtained by submerged cultivation of a microorganism of genus Penicillium or Eupenicillium producing mycophenolic acid.

3. The process of claim 1, wherein the suitable base is sodium or potassium hydroxide

4. The process of claim 1, wherein the suspension with pH from 9 to 13 is stirred for at least one hour.

5. The process of claim 1, wherein the separation of the mycelium from the liquid phase is accomplished by filtration.

6. The process of claim 1, wherein the suitable acid is sulfuric acid, phosphoric acid or hydrochloric acid.

7. The process of claim 1, wherein the pH of the aqueous suspension of mycophenolic acid is from about 4.5 to about 1.5.

8. The process of claim 1, wherein the separation of mycophenolic acid from the liquid phase is accomplished by filtration.

9. The process of claim 1, wherein the separated mycophenolic acid is dried prior to dissolution in toluene.

10. The process of claim 1, wherein the mycophenolic acid with purity higher than 99.0% is obtained by crystallization from toluene.
